(19) United States
(12) Patent Application Publication
(10) Pub. No.: US 2009/0098596 A1
(43) Pub. Date: Apr. 16, 2009

(54) PHOTOSTABLE FRET-COMPETENT BIARSENICAL-TETRACYSTEINE PROBES BASED ON FLUORINATED FLUORESCEINS

(75) Inventors: Elisabeth A. Jares-Erijman, Buenos Aires (AR); Carla C. Spagnuolo, Buenos Aires (AR); Rolf J. Vermeij, MH Glanerburg (NL); Thomas M. Jovin, Goettingen (DE)

Correspondence Address: PATENT CENTRAL LLC;Stephan A. Pendorf, 1401 Hollywood Boulevard, Hollywood, FL, 33020, US

(73) Assignee: Max-Planck Gesellschaft zur Foerderung der Wissenschaften e.V., Muenchen (DE)

(21) Appl. No.: 12/332,450

(22) Filed: Dec. 11, 2008

(30) Foreign Application Priority Data
Jun. 12, 2006 (EP) 06012026

(51) Int. Cl. C12Q 1/02; C07D 311/82; C07K 14/00

(52) U.S. Cl. 435/29; 549/388; 530/350

(57) ABSTRACT

A fluorogenic dye having the formula

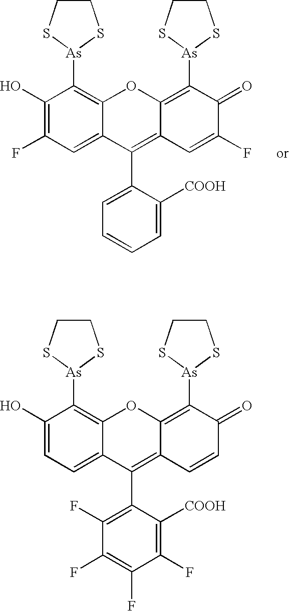

and salts of said fluorogenic dyes.

### FIELD OF THE INVENTION

The invention relates to fluorogenic dyes, complexes of such fluorogenic dyes and a protein with a tetracysteine motif, a method of synthesizing such fluorogenic dyes, a method of examining a sample using such fluorogenic dyes, and a use of such fluorogenic dyes.

### RELATED ART OF THE INVENTION

Reference is made to the following documents of related art:
(1) Griffin, B. A.; Adams, S. R.; Tsien, R. Y., Science 1998, 281, 269-272.
(2) Sosinsky, G.; Gaietta, G.; Giepmans, B.; Deerinck, T.; Adams, S.; Hand, G.; Mackey, M.; Terada, M.; Smock, A.; Tsien, R. Y.; Ellisman, M., Biophys. J 2005, 88, 31A-32A.
(3) Tour, O.; Meijer, R. M.; Zacharias, D. A.; Adams, S. R.; Tsien, R. Y., Nat. Biotechnol. 2003, 21, 1505-1508.
(4) Hoffmann, C.; Gaietta, G.; Bunemann, M.; Adams, S. R.; Oberdorff-Maass, S.; Behr, B.; Vilardaga, J. P.; Tsien, R. Y.; Eisman, M. H.; Lohse, M. J., Nat. Methods 2005, 2, 171-176.
(5) Jares-Erijman, E. A.; Jovin, T. M., Nat. Biotechnol. 2003, 21, 1387-1395.
(6) Selvin, P. R., Annu. Rev. Biophys. Biomol. Struct. 2002, 31, 275-302.
(7) Martin, B. R.; Giepmans, B. N. G.; Adams, S. R.; Tsien, R. Y., Nat. Biotechnol. 2005, 23, 1308-1314.
(8) Adams, S. R.; Campbell, R. E.; Gross, L. A.; Martin, B. R.; Walkup, G. K.; Yao, Y.; Llopis, J.; Tsien, R. Y., J. Am. Chem. Soc. 2002, 124, 6063-6076.
(9) Sun, W. C.; Gee, K. R.; Klaubert, D. H.; Haugland, R. P., J. Org. Chem. 1997, 62, 6469-6475.
(10) Lindqvist, L., Arkiv. Kemi 1961, 16, 79-138.
(11) Sun, W. C.; Gee, K. R.; Klaubert, D. H.; Haugland, R. P., Synthesis of fluorinated fluoresceins. J. Org. Chem. 1997, 62, (19), 6469-6475.
(12) Adams, S. R.; Campbell, R. E.; Gross, L. A.; Martin, B. R.; Walkup, G. K.; Yao, Y.; Llopis, J.; Tsien, R. Y., New biarsenical Ligands and tetracysteine motifs for protein labeling in vitro and in vivo: Synthesis and biological applications. J. Am. Chem. Soc. 2002, 124, (21), 6063-6076.
(13) Jovin, T. M.; Arndt-Jovin, D. J.; Marriott, G.; Clegg, R. M.; Robert-Nicaud, M.; Schormnann, T. Optical Microscopy for Biology 1990, 575-602.

Biarsenical ligands are membrane-permeable fluorogenic dyes, which form highly stable complexes with tetracysteine motifs engineered in a target protein of interest.¹ The probes and targets are small compared to visible fluorescent proteins, thereby reducing potential stereochemical interference while providing (i) controllable times of delivery in pulse chase experiments,² and (ii) special reactivities enabling chromophore assisted light inactivation experiments (CALI)³ and non-fluorescent readouts.² Furthermore, the combination of biarsenical dyes with visible fluorescence proteins (VFPs) as Forster resonance energy transfer⁴ donor-acceptor (DA) pairs constitutes a very attractive technology for the assessment of conformational changes and molecular interactions in living cells. Due to the inverse 6^{th} power distance dependence of FRET, the range of separations that can be determined with confidence about the Forster critical distance for 50% FRET efficiency (R₀) is very narrow.^{5,6} The generation of DA pairs with large R₀ is essential for extending the range over which FRET is operative.

Although optimization of the biarsenical binding motif has led to significant improvements in affinity and signal levels,⁷ the limited photostability and pH sensitivity of fluorescein derivatives in the physiological range constitute inherent limitations that still preclude their widespread application.

It is an object of the current invention to provide improved fluorogenic dyes. More specifically, it is an object of the current invention to provide an improved biarsenical tetracyctein probe exhibiting significant improvements in important properties over the original fluorescein derivative F1AsH, in particular a higher absorbance, larger Stokes shift, higher quantum yield, higher photostability, and reduced pH dependence.

It is a further object of the invention to provide a suitable method of synthesizing such improved dyes.

It is a still further object of the current invention to provide an examining method using such improved dyes.

It is a still further object of current invention to provide an advantageous use for such improved dyes.

### SUMMARY OF THE INVENTION

In one aspect of the invention there is provided a fluorogenic dye having the formula

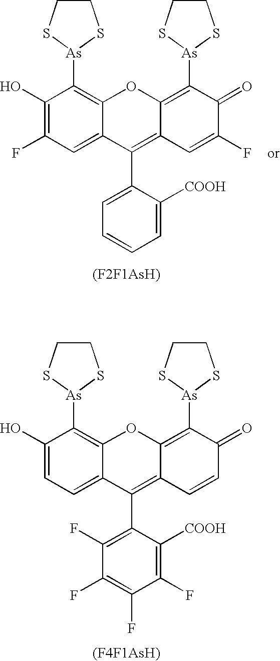

and salts of said fluorogenic dyes.

The invention introduces fluoro-substituted versions, F2F1AsH and F4F1AsH, exhibiting significant improvements in important properties over the original fluorescein derivative F1AsH⁸. Compared to F1AsH, F2F1AsH has higher absorbance, larger Stokes shift, higher quantum yield, higher photostability, and reduced pH dependence. The emission of F4F1AsH lies in a region intermediate to that of F1AsH and ReAsH (a resorufin biarsenical),⁸ providing a new color and excellent luminosity. In addition, the two new probes form a new FRET pair with a substantially larger R₀ value than any obtained with these dyes (see below).

Syntheses of the tetrafluorinated (F4F1AsH-EDT₂) and difluorinated F1AsH (F2F1AsH-EDT₂) derivatives most advantageously is accomplished from the parent halogenated fluoresceins according to⁴. In particular the synthesis comprises the steps of
reacting

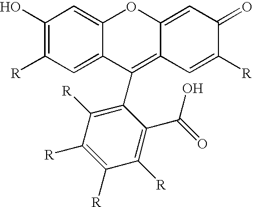

with HgO to form

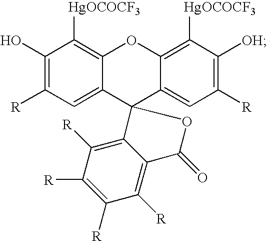

reacting

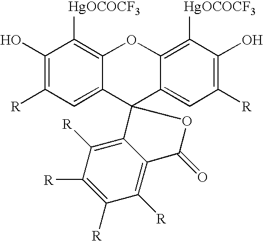

with AsCI₃ to form

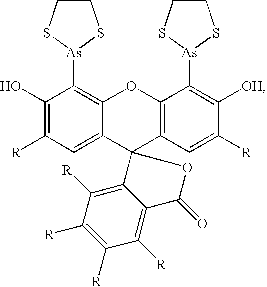

wherein R=H, F. No reduction step is required to give the —COOH group, that form is always present in the original compound, which is in an equilibrium between the two forms.

The absorption maximum of F2F1AsH is shifted 11 nm to the blue, compared to F1AsH, whereas the maximum of F4F1AsH is displaced 17 mn to the red (FIG. 1, Table 1). Upon formation of a complex of F2F1AsH with a 12-mer peptidic sequence (FLNCCPGCCMEP, P12) as a model target,⁷ fluorescence is observed with an emission peak at 522 nm. Thus, the Stokes shift is 22 nm, 7 nm greater than that of the F1AsH complex. The fluorescence intensity (λ_{exc} 490 nm, λₑₘ 525 nm) is 4-fold that of the complex with the parent F1AsH probe. This enhancement is attributable to a larger extinction coefficient at 490 nm (2×), and a greater emission quantum yield (2×). The radiative lifetime of F2F1ASH-P12 (4.78 ns) is similar to that of the corresponding F1AsH complex (4.88 ns, Table 1).

The emission peak of F4F1AsH-P 12 at 544 nm expands the spectral range of the biarsenical dyes. In addition, the fluorescence lifetime increases to a value (5.2 ns). The two fluorinated derivatives provide new combinations with FRET donors and acceptors within and outside of the biarsenical family.

TABLE 1
Photophysical data for the biarsenical-P12 complexes

| | λ_{abs} | λₑₘ | εₘₐₓ | τ | k_{bl} | pb^{a} |
|---|---|---|---|---|---|---|
| | [nm] | [nm] | [M⁻¹ cm⁻¹] | [ns] | [s⁻¹] | [%] |
| FlAsH-P12 | 511 | 527 | 52000 | 4.88 | 3.2 · 10⁴ | 87 |
| F2FlAsH-P12 | 500 | 522 | 65500 | 4.78 | 6.2 · 10² | 32 |
| F4FlAsH-P12 | 528 | 544 | 35100 | 5.18 | 9.1 · 10³ | 89 |

^{a}photobleaching: loss of fluorescence after 120 min of irradiation.

It has been reported that fluorination of fluorescein⁹ leads to greater resistance to photobleaching to a degree that depends on the number of fluorine atoms. The inventors observed a 50× increase in photostability of the 2′,7′-difluoroderivative, whereas for the tetrafluoroderivative, the photostability is similar to that of the parent dye-complex (FIG. 2, Table 1). The fluorine atoms in positions 2′ and 7′, presumably lead to a reduction in lifetime of the triplet state that serves as an intermediate in the photobleaching process.¹⁰

To evaluate the sensitivity to pH, the probes were dissolved in phosphate buffers ranging in pH from 7.8 to 5.6. F1AsH-P12 displayed a 50% decrease at the absorption peak of the dianion responsible for fluorescence, whereas the absorption of F2F1AsH-P12, and F4F1AsH-P12 only decreased by 16% (Figure S2; Supporting information). The complexes of the fluorinated dyes exhibited a corresponding brighter emission at lower pH. These results were expected in view of the lower pKs of the dianion and monoanion forms of the parent fluorinated fluoresceins.⁹

The R₀ values of F2F1AsH, F4F1AsH, F1AsH and ReAsH in different donor-acceptor combinations are given in Table 2. F2F1AsH and F4F1AsH have a very favorable spectral overlap, leading to a large J (the overlap integral) and thus an R₀ of 5.4 nm, greater than any of the values obtained with the other combinations of donors and acceptors based on biarsenical dyes. For example the F1ASH-ReAsH pair has an R₀ of 3.9 nm. Thus, the new F2F1AsH-F4F1AsH probes constitute a new donor acceptor pair extending the dynamic range for heteroFRET in studies of living cells by 40%. They also provide new possibilities in combination with other FRET donors and acceptors within and outside the biarsenical family.

TABLE 2
Critical Förster distances

Acceptor

| | ReAsH | | F4FlAsH | |
|---|---|---|---|---|
| Donor | 10¹⁴ · J | Rₒ nm (Φ_{d})^{a} | 10¹³ · J | Rₒ nm (Φ_{d}) |
| FlAsH | 5.03 | 3.9 (0.4)^{b} | 1.48 | 4.7 (0.4) |
| F2FlAsH | 3.44 | 4.1 (0.8) | 1.58 | 5.4 (0.8) |
| F4FlAsH | 6.45 | 4.1 (0.4) | 1.17 | 4.5 (0.4) |
| | F2FlAsH | | FlAsH | |
| | 10¹³ · J | Rₒ nm (Φ_{d}) | 10¹⁴ · J | Rₒ nm (Φ_{d}) |
| F2FlAsH | 1.37 | 5.2 (0.8) | 6.41 | 4.1 (0.4) |

^{a}Emission quantum yield.
^{b}Approximate value from⁸.

The F2F1AsH-F4F1AsH DA pair was employed in a titration of a F2F1AsH complex of biotin-P12 bound to streptavidin, with the same peptide bearing F4F1AsH (FIG. 3). Upon saturation of the free sites remaining on the tetrameric streptavidin, the FRET efficiency was 0.34, corresponding to an apparent mean computed transfer distance of 5.0 nm.

An important consideration related to quantitative FRET determinations based on VFPs is the restricted motion of the chromophore inside the β-barrel of the protein. In order to accurately estimate distances by FRET, one requires knowledge of the relative orientation of the dyes. The general assumption of the value 2/3 for the orientation factor κ² only applies if both donor and acceptor are in rapid, isotropic rotational motion. This is impossible for VFPs due to their mass (27 Kda); the rotational correlation times are much longer than the fluorescence lifetimes.

The fluorescence anisotropies determined for F2F1AsH-P 12 biotin, and F4F1AsH-P12 biotin (1 μM in 20 mM HEPES, pH 7.4) were 0.038, and 0.046, respectively, implying that one can apply the 2/3 κ² value with confidence in the case of small and/or mobile targets, thereby allowing accurate distance determinations by FRET. Rotational motion may be restricted in larger proteins, thereby enabling FRET measurements by homotransfer.⁵

In conclusion, we present two new derivatives of the F1AsH family, one of them with 50× improved photostability, lower pH sensitivity, higher absorbance and quantum yield, and the second adding a new color to the palette of biarsenical dyes. In addition, the two compounds form an excellent FRET pair with a large critical distance, facilitating improved structural and dynamic studies of living cells.

### BRIEF DESCRIPTION OF THE DRAWINGS

To provide a better understanding of the current invention materials and methods are explained below in detail. Further, for better illustration some drawings are provided in which

FIG. 1 illustrates dye structures, and absorption and emission (□) spectra of F2F1AsH-P12 (−) and F4F1AsH-P 12 (- -);

FIG. 2 illustrates a graph illustrations photobleaching of F2F1AsH-P12 (◯), F4F1AsH-P12 (□) and F1AsH-P12 (+). Full lines correspond to exponential fits. Complexes of the three dyes (10 μM) were irradiated with a mercury arc lamp through a 490-560 nm filter with an irradiance of 70 mW/cm²;

FIG. 3 illustrates titration of a complex of streptavidin (0.8 μM)-biotinylated F2F1AsH-P12 (0.8 μM), with biotinylated F4F1AsH-P12 (spectrally unmixed data). Inset: relative change in donor emission (at 520 nm);

FIG. 4 illustrates fluorescence emission spectra of biarsenical-peptide complexes (0.1 μM). F2F1AsH-P12 (λ_{exc} 490 nm); F4F1AsH-P12 (λ_{exc} 520 nm); F1AsH-P12 (λ_{exc} 490 nm);

FIG. 5 illustrates the dependence of the absorption (left panel) and emission (right panel) properties within the physiological pH range (5.7-7.8); and

FIG. 6 illustrates photobleaching recorded during 120 min. of irradiation and monitored by fluorescence emission. λ_{exc} 490 nm. a. F1AsH-P12; b. F2F1AsH-P12; c. F4F1AsH-P12.

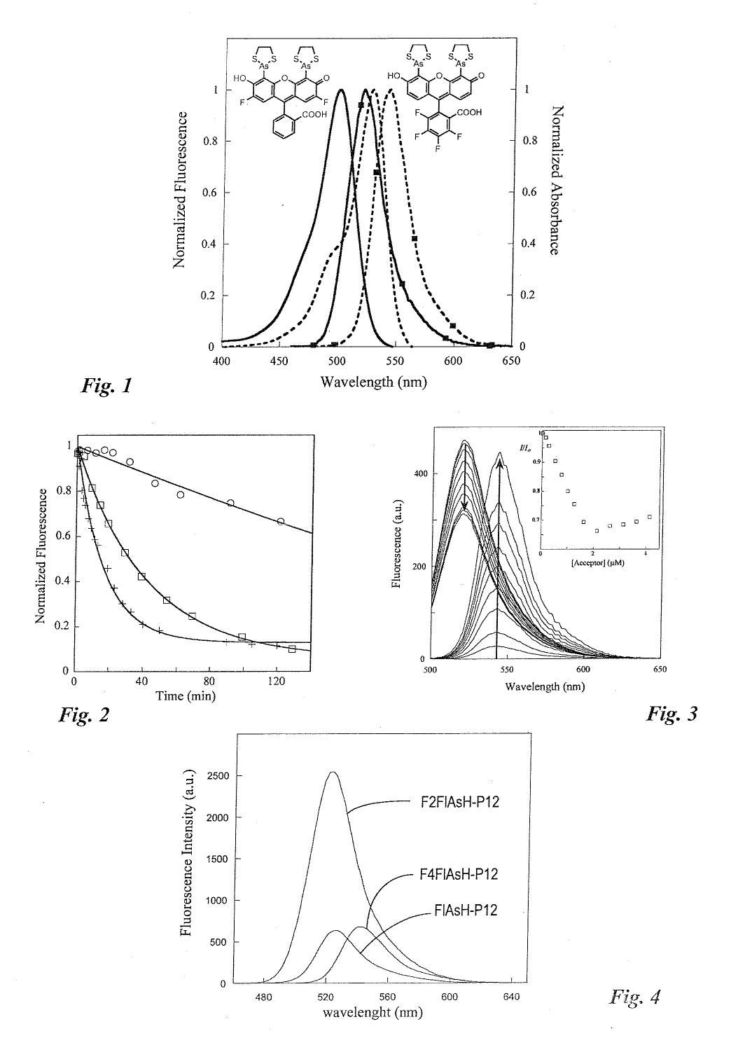

### DETAILED DESCRIPTION OF THE INVENTION

### 1-Materials

General Procedures. NMR measurements were carried out on a Bruker 200 MHz AM, 400 MHz, 500 MHz AMX, Mercury 300 MHz (Varian) or on a INOVA 500 MHz (Varian) NMR spectrometer. Chemical shifts are in ppm (internal reference TMS) and coupling constants are given in Hz.

4-Fluororesorcinol was purchased from CPC Scientific, San Jose, Calif., USA and model peptide from WITA GmbH, Germany. All other chemicals were obtained from Aldrich Chem. Co.

Reactions were monitored by thin-layer chromatography on Merck silica gel plates (60F-254). Column chromatography was performed on silica gel (230-400 mesh, Merck ASTM) or on Fluorisil® (60-100 mesh, J. T. Baker).

ESI and HRMS were measured on an APEX IV 7 Tesla-Fourier Transform Ion Cyclotron Resonance (FTICR)-Mass spectrometer (Bruker) or on a TSQ 7000 Triple-Stage-Quadrupol-Instrument (Finnigan) with Electrospray-Ionisation, at the Georg-August-University of Goettingen, Germany.

Absorption and fluorescence spectra were measured with a UVIKON 943 Double Beam UV/Vis absorption spectrophotometer and with a Perkin Elmer LS50B fluorescence spectrophotometer, respectively. Lifetime measurements were made by TCSPC in a Horiba Jobin Yvon IBH Fluorescence Lifetime Spectrometer System. The excitation source at 495 nm was a NanoLED N-01 Aqua and the detection module was TBX-04-A. Irradiation was carried out using a Superlite SUV-DC-P system incorporating a 200W DC Super-Pressure short arc lamp coupled to a light guide for high UV transmission and an electronic timer for exposure time control (Lumatec GmbH,

### 2-Synthetic Methods

### General Synthesis of Biarsenical Derivatives

3,4,5,6-tetrafluorofluorescein, 2′,7′-difluorofluorescein and F1AsH derivatives were prepared according to ^{11,12}.

Fluorescein-4′,5′-bis(mercuric diacetate). Fluorescein (1 g, 3 mmol) was added to a stirred solution of HgO (2.6 g, 12 mmol) in trifluoroacetic acid (40 mL). The reaction was mantained at room temperature overnight, added to water and the precipitate was collected by filtration and dried in vacuo over P₂O₅. Yield, 2.5 g (87%). The crude product was used without further purification.

2′,7′-difluorofluorescein-4′,5′-bis(mercuric diacetate). 2′,7′-difluorofluorescein (0.3 g, 0.8 mmol) was added to a stirred solution of HgO (0.37 g, 1.7 mmol) in trifluoroacetic acid (6.5 mL). The reaction was mantained at room temperature overnight, added to water and the precipitate was collected by filtration and dried in vacuo over P₂O₅. Yield, 0.65 g (80%). The crude product was used without further purification.

3,4,5,6-tetrafluorofluorescein-4′,5′-bis(mercuric diacetate). 3,4,5,6-tetrafluorofluorescein (0.5 g, 1.13 mmol) was added to a stirred solution of HgO (0.52 g, 2.4 mmol) in trifluoroacetic acid (9 mL). The reaction was mantained at room temperature overnight, the precipitate was collected by filtration and dried in vacuo over P₂O₅. Yield, 1.10 g (95%). The crude product was used without further purification.

4′,5′-Bis(1,2,3-dithioarsolan-2-yl)-fluorescein, F1AsH-EDT₂. Crude Fluorescein-4′,5′-bis(mercuric trifluoroacetate) (92.3 mg, 0.1 mmol) was suspended in dry NMP (1.5 mL) under Ar and treated with arsenic trichloride (0.16 mL, 2.0 mmol), DIPEA (0.14 mL, 0.80 mmol), and palladium (II) acetate (1 mg). The reaction mix was kept overnight at room temperature and followingly poored on aqueous phosphate buffer, pH 7: acetone (1:1 v/v 50 mL, 0.25 M K₂HPO₄), stirred for 5 min, and additioned with ethanedithiol (0.5 mL). After 20 min of stirring, CHCl₃ (30 mL) and acetic acid were added to acidify the aqueous phase, and the mixture was stirred for 1 h before the phases were separated. The aqueous layer was extracted (2×30 mL) with CHCl₃. The combined organic layers were dried over Na₂SO₄ and evaporated to dryness. The orange residue was purified by chromatography on Silica Gel (Packed in with CHCl₃-0.5% HOAc, sample loaded in CHCl₃) with elution from 0.5% HOAc-CHCl₃ to ethyl acetate-0.5% HOAc. The combined fractions were evaporated and subjected to trituration with EtOH-H₂O. 22.5 mg (34% yield) of a whitish-pink solid was obtained. ¹H-NMR (CDCl₃-CD₃OD 1:1, ppm): 3.54 (m, partially obscured by solvent), 6.49 (d, 2H, J=9 Hz), 6.62 (d, 2H, J=9 Hz), 7.20 (d, J=6 Hz, H-6), 7.65 (dd, J=6 and 2 Hz, H-4,5), 8.00 (d, J=6 Hz, H-3), 9.89 (s, 2H, OH). ESI-HRMS: [M+H]⁺: 664.8541. Calcd for C₂₄H₁₉O₄As₂S₄ 663.8547.

4′,5′-Bis(1,2,3-dithioarsolan-2-yl)-2′,7′-difluorofluorescein, F2-F1AsH-EDT₂. Crude 2′,7′-difluoroFluorescein-4′,5′-bis(mercuric trifluoroacetate) (0.2 g, 0.2 mmol) was suspended in dry NMP (3 mL) under Ar and treated with arsenic trichloride (0.34 mL, 4.0 mmol), DIPEA (0.28 mL, 1.6 mmol), and palladium (II) acetate (1 mg), following the procedure described above. Purification was carried out by column chromatography packed with Florisil, eluted with CH₂Cl₂-0.5% HAcO gradient up to AcOEt-0.5% HAcO. The combined fractions were evaporated and subjected to trituration with EtOH-H₂O. 13.4 mg (9.8% yield) of an orange solid was obtained. ¹H-NMR (CDCl₃, ppm): 3.61 (m, 8H), 6.42 (d, 2H, J=10 Hz ), 7.21 (d, 1H, J=5 Hz, H-3), 7.65-7.74 (m, 2H, H-4, 5), 8.03 (d, 1H, J=5 Hz, H-6), 10.13 (s, 2H, OH). ES-HRMS [M+H]⁺: 700.8358 Calcd for C₂₄H₁₇F₂O₅As₂S₄ 700.8358.

4′,5′-Bis(1,2,3-dithioarsolan-2-yl)-3,4,5,6-tetrafluorofluorescein, F4-Flash-EDT₂. Crude 3,4,5,6-tetrafluoroFluorescein-4′,5′-bis(mercuric trifluoroacetate) (0.3 g, 0.3 mmol) was suspended in dry NMP (3 mL) under Ar and treated with arsenic trichloride (0.5 mL, 3.0 mmol), DIPEA (0.4 mL, 2.4 mmol), and palladium (II) acetate (1 mg), following the procedure described above. Purification was carried out by chromatography on a Florisil column, eluted with CH₂Cl₂-0.5% HAcO gradient up to AcOEt-0.5% HAcO. The combined fractions were evaporated and subjected to trituration with EtOH-H₂O. ¹H-NMR (CDCl₃-CD₃OD, 1:1, ppm): 3.49 (m, partially obscured by solvent), 7.06 (d, 2H, J=8.6 Hz), 7.25 (d, 2H, J=8.6 Hz), 10.50 (s, 2H, OH). ES-HRMS: [M+H]⁺: 736.8168. Calcd for C₂₅H₁₅As₂F₅O₅S₄ 736.8170.

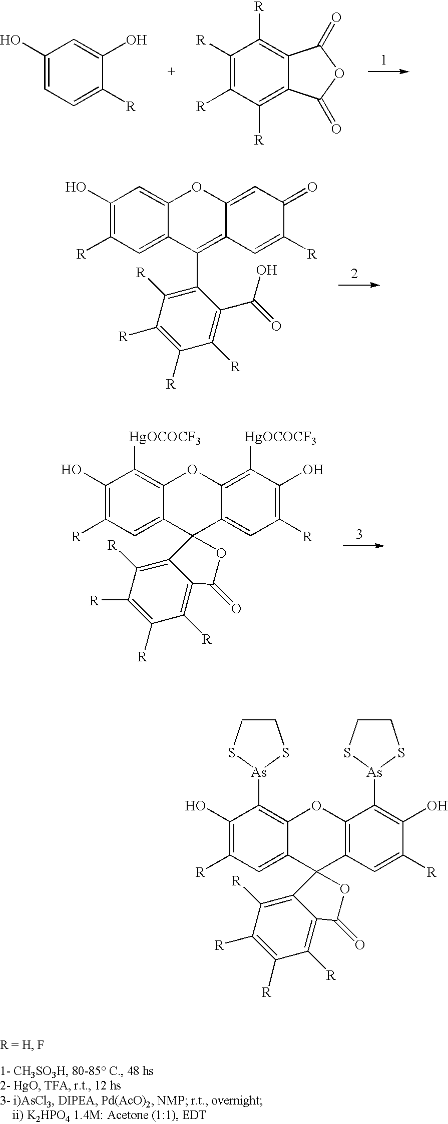

### 3-Spectroscopic Properties

3.1-In vitro Labeling of a Model Peptide with Fluorinated F1AsH Derivatives

To a solution of the biarsenical compound in buffer HEPES 20 mM, 1 mM 2-ME, pH 7.4, at a concentration of 0.1 μM and with a slight excess of EDT, was added the model target peptide P12 (FLNCCPGCCMEP) to a final concentration of 1 μM from a stock solution in the same buffer and treated with 10 mM TCEP to reduce any disulfide bond. Emission spectra were taken after two hours of formation of the complex. FIG. 4 depicts the emision spectra of the F2F1AsH, F4F1AsH and F1AsH at equal concentration, with excitation at 490 nm. A 4-fold enhanced fluorescence emission of F2F1AsH compared to F1AsH can be observed.

### 3.2-Absorption Spectra of Biarsenical Compounds and pH Dependence in the Physiological Range

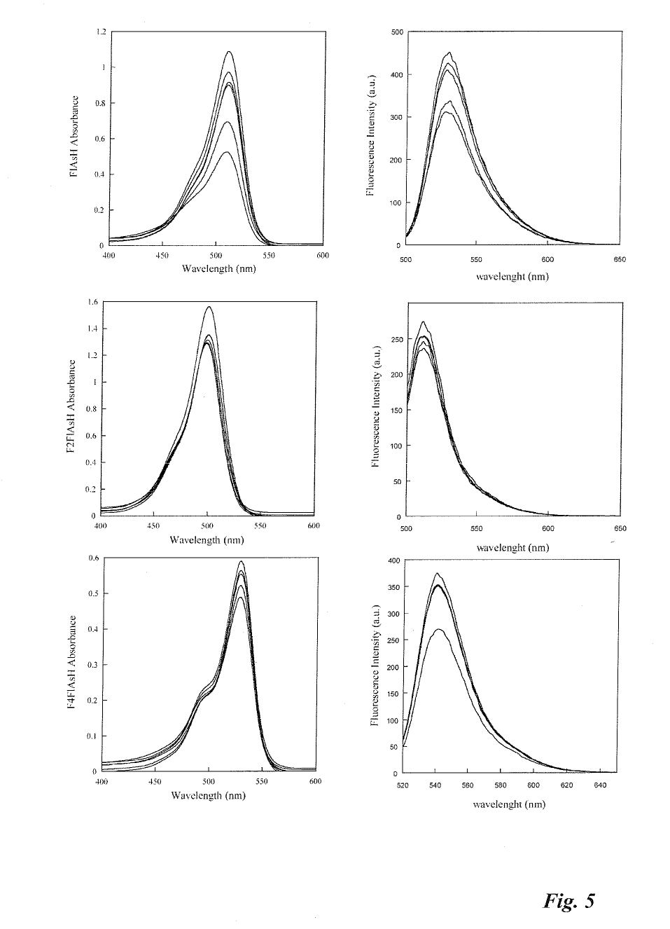

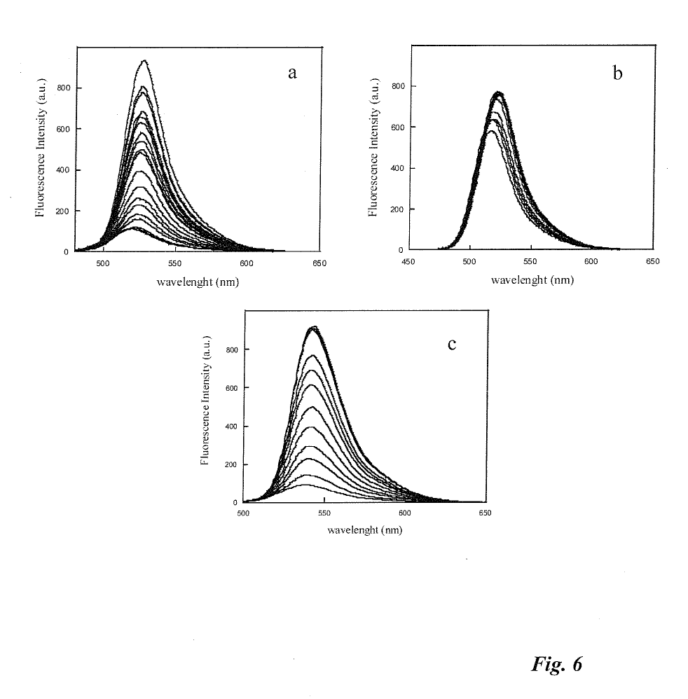

The absorption spectra of 20 μM solutions of F2F1AsH, F4F1AsH and F1AsH-EDT₂, in 20 mM phosphate buffer, at different pH values, are shown in FIG. 5. In all cases, the absorbance and the corresponding emission, increases with the increase of pH.

### 3.3—Fluorescence Lifetimes

The fluorescence lifetimes of the complexes obtained as described in section 3.1, were determined by TCSPC. The corresponding values for each parent fluorescein were also determined in 20 mM buffer HEPES, pH 7.4 for comparison.

| P12-Complex | t (ns) | Fluorophore | t (ns) |
|---|---|---|---|
| FlAsH | 4.88 | Fluorescein | 4.07 |
| F2FlAsH | 4.78 | 2′,7′-difluorofluorescein | 4.02 |
| F4FlAsH | 5.18 | 3,4,5,6-tetrafluorofluorescein | 4.13 |

### 4-Photostability

Samples containing 10 μM of the biarsenical compounds and 10 μM of the model peptide P12, were incubated at room temperature for 1 h. Each solution of the F1AsH-peptide complex was irradiated for 120 min as previously described (see General Procedures).

Values of κ_{bl} were calculated from the monoexponential fitting of the experimental photobleaching decay, according to the model described in (Eq. 1) for the intermediate irradieation regime.¹³

k_{fit}=k_{bl}·τ·Ψ·σ·t Eq. 1

where τ(s) is the lifetime, Ψ is the irradiance (photons cm⁻² s⁻¹) and σ is the molecular absorption cross-section (cm² molecules⁻¹). Inasmuch as a broad excitation bandwidth was used, σ was computed as a mean value over the spectral distribution function of photon flux and corrected by the spectral integral with the corresponding absorption spectra, including the irradiation source and filter.

Photobleaching of F2F1AsH, F4F1AsH and F1AsH complexes with P12 lead to a residual fluorescence with maxima at 516, 538 and 519.5 nm, respectively.

### 5-FRET Determinations

A 0.8 μM solution in 20 mM buffer HEPES, 0.1 M NaCl, 1 mM 2-ME, pH 7.4 of the complex F2F1AsH-P12-biotin was incubated with an equimolar solution of streptavidin and titrated with increasing amounts of F4F1AsH-P 12-biotin in a quartz cuvette equipped with a magnetic stirrer.

Each addition was followed by measurement of the emission intensity using 490 nm as the excitation wavelength.

FRET efficiency was determined according to.

### 6-Steady State Anisotropy

Steady state emission anisotropy of 1 μM solutions of F2F1AsH-P12-biotin and F4F1AsH-P12-biotin in 20 mM in buffer HEPES, NaCl 0.1M, 1 mM 2-ME, pH 7.4 was determined abased on eq. 2, were G is the correction factor for the bias in the detection of the two polarized components I_{□}, I_{⊥}

$$r = \frac{( {I}_{•} / {I}_{⊥} ) - G}{{I}_{•} / {I}_{⊥} + 2 G} \qquad Eq . 2$$

Now that the invention has been described,

1. A fluorogenic dye having the formula 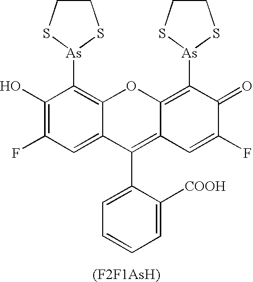
and salts of said fluorogenic dye.

2. A fluorogenic dye having the formula 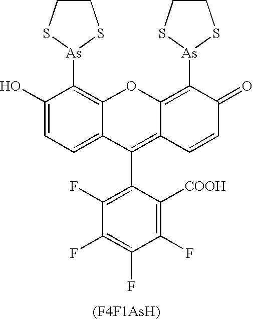
and salts of said fluorogenic dye.

3. A complex of the fluorogenic dye of claim 1 and a protein with a tetracystein motif.

4. (canceled)

5. A method for examining a sample comprising the steps
contacting the fluorogenic dye of claim 1 with a sample
measuring fluorescence.

6. The method of claim 5, wherein the sample comprises a protein.

7. The method of claim 5, wherein the sample is a living biological cell.

8. (canceled)

9. (canceled)

10. A method of synthesizing the fluorogenic dye of the formula: 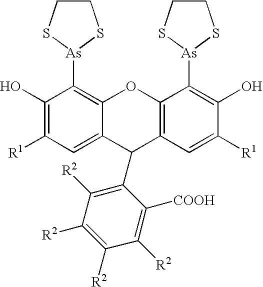
wherein R¹ and R²=F or H, and when R¹=F, R²=H, and when R¹=H, R²=F, and salts of said fluorogenic dye, said method comprising the steps of
a) reacting 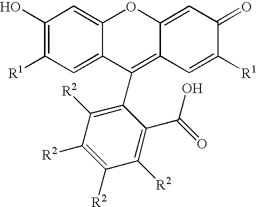
with HgO to form 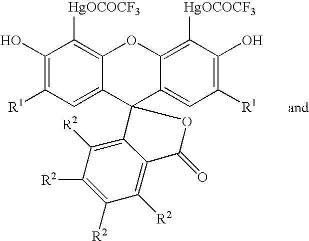
b) reacting 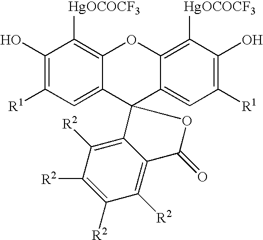
with AsCl₃ and EDT to form

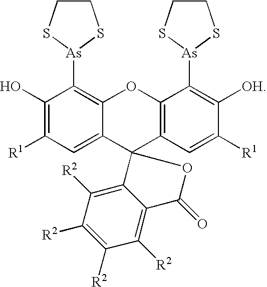

11. A method for conducting a fluorescence resonance energy transfer (FRET) experiment, comprising
a) contacting a sample with at least one fluorogenic dye having the formula 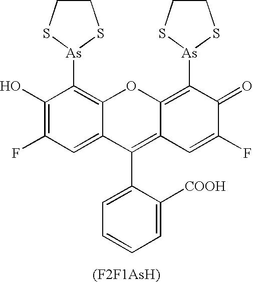
and salts of said fluorogenic dye, or 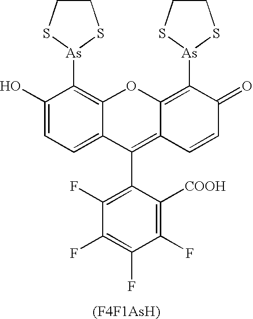
and salts of said fluorogenic dye, and
b) measuring the fluorescence of the sample.

12. The method of claim 8, wherein both (F2F1AsH) and (F4F1AsH) are involved and forming a FRET pair, one dye acting as a FRET donor and the other dye acting as a FRET acceptor.
